(19) United States
(12) Patent Application Publication
(10) Pub. No.: US 2024/0350796 A1
(43) Pub. Date: Oct. 24, 2024

(54) NEUROIMPLANT WITH DATA RATE REDUCTION SECTION

(71) Applicant: CorTec GmbH, Freiburg (DE)

(72) Inventors: Stefan REICH, Munich (DE); Maurits ORTMANNS, Ulm (DE); Joachim BECKER, Beimerstetten (DE); Martin SCHUETTLER, Emmendingen (DE); Stefan RIEGER, Freiburg (DE)

(21) Appl. No.: 18/763,739

(22) Filed: Jul. 3, 2024

Related U.S. Application Data
(63) Continuation of application No. PCT/EP2023/075186, filed on Sep. 13, 2023.

(30) Foreign Application Priority Data
Sep. 15, 2022 (DE) 10 2022 123 704.4

(51) Int. Cl. A61N 1/02; A61N 1/05

(52) U.S. Cl. CPC A61N 1/025; A61N 1/0529

(57) ABSTRACT

A neuroimplant for the brain region is provided. The neuroimplant has at least one detection section for detecting signals from the brain region, an electrode section having at least one electrode which can be coupled to the detection section and can be arranged in the brain region and is designed for electrically contacting the brain region, at least one digitization section for generating at least one digital data stream from the signals detected by the at least one detection section, where the at least one data stream in each case includes a sequence of data points, and a data reduction section which is designed to discard data points in the sequence of data points from the at least one data stream according to a predetermined scheme.

### CROSS REFERENCE TO RELATED APPLICATIONS

This application is a continuation of International Application No. PCT/EP2023/075186, filed Sep. 13, 2023, which takes priority from German Patent Application No. 10 2022 123 704.4, filed on Sep. 15, 2022, the contents of which are incorporated by reference herein.

### FIELD OF THE INVENTION

The present invention relates to a neuroimplant with a data rate reduction section.

### BACKGROUND

Neuroimplants are used in human-machine interfaces. These are based on implantable microelectronics which allow low-noise readout of neural signals in parallel on a plurality of channels and flexible stimulation of brain activity.

Medical applications include the diagnosis and treatment of neurodegenerative diseases such as Parkinson's, Alzheimer's and epilepsy. In addition, applications in basic neurophysiological research and in the field of novel, directly thought-controlled prostheses are conceivable.

The wide variety of applications also fundamentally results in different requirements for the underlying hardware. For example, in implantable systems, the allowable power consumption is severely limited because the surrounding tissue could be damaged by excessive heat development. The neuronal signals of interest are basically divided into two categories. In the low frequency range between about 0.2 and 200 Hertz, local field potentials can be measured, which are composed of the superimposed potential of hundreds or even thousands of neurons in the region around the recording electrode. The second category comprises the action potentials (spikes) that occur in the frequency range from about 200 to 8000 Hertz and represent the activation process of a single neuron. Depending on the application, different signal bands and different numbers of channels are desired as a data source. At the same time, however, it is desirable to provide a common hardware platform for all applications so that the research in this area is not slowed down by the limited availability of precisely tailored microelectronics.

### SUMMARY

It is therefore an object of the present invention to provide a data rate module for a neuroimplant by means of which the aforementioned technical problems are at least partially alleviated.

This object may be achieved by a data rate module in accordance with this disclosure.

Accordingly, a neuroimplant for the brain region may be provided, comprising:
at least one detection section for detecting signals from the brain region,
an electrode section having at least one electrode which can be coupled to the detection section and can be arranged in the brain region and is designed for electrically contacting the brain region,
at least one digitization section for generating at least one digital data stream from the signals detected by the at least one detection section,
wherein the at least one data stream in each case comprises a sequence of data points,
and
a data reduction section designed to discard data points in the sequence of data points from the at least one data stream according to a predetermined scheme.

The data rate of the at least one data stream can thus be reduced according to the predetermined scheme.

The adjustment of the data rate at which neural signals are output can be specifically controlled via two schemes that make the selection of channels of interest and the number of data points to be discarded between two transmitted data points per channel flexibly adjustable. Adaptation (=optimization) of a common hardware platform is thus possible in a wide variety of applications.

The innovation, which is the subject matter of this disclosure, accordingly comprises a digital section which allows the data rate of the chip to be modified using very simple means. This has direct effects both on the utilization of the reading instance (e.g., microcontroller) and on the power consumption of the system. Both can be reduced. Power reductions are of the utmost importance in particular for implantable microelectronics.

Moreover, optimizations for certain application scenarios are possible with simultaneous use of an identical hardware platform.

In this case, the scheme can comprise discarding all data points of a data stream.

Alternatively or additionally, the scheme may comprise regularly discarding a predetermined number of data points from the at least one data stream.

In general, the scheme may comprise discarding n data points from each sequence of m data points of a data stream, where m and n are natural numbers.

A first register may be provided, which contains a value for each of the at least one data streams, which is representative of whether all data points of the respective data stream are discarded or not.

A second register may be provided which contains a value representative of discarding n data points from each sequence of m data points of at least one data stream.

In this case, the first and/or the second register can be programmable.

In the neuroimplant according to one variant, it is provided that the at least one detection section, the at least one digitization section and the data reduction section are arranged on a common semiconductor substrate and, in particular, are designed as an ASIC chip.

In one variant, the section for reducing the output data rate specifically provides two schemes:
channel selection: by setting a, for example, 32-bit register, it is individually determined for each channel whether the measured data are discarded (“0”) or not (“1”), i.e., output.
discarding data points: by setting a, for example, 5-bit register, a number is defined which determines how many available data points per channel are discarded between two data points actually sent. For example, a “1” (binary “00001”) means that one data point is discarded between two output data points—thus only every second data point leaves the chip, and the data rate is halved accordingly.

Both schemes can be combined without the system becoming untestable and thus unverifiable due to the large number of setting options. This is an important feature, as very strict approval restrictions must be met for human-implantable electronics.

In particular, discarding the data points that are not required allows a significant increase in efficiency for systems that only require local field potentials and can therefore dispense with the higher data rate required to measure spike potentials. Averaging to nevertheless utilize the discarded data points was deliberately dispensed with, since in general no significant improvement in the input-related noise is to be expected. This is due to the fact that the flicker noise (1/f noise) is the dominant noise process, in particular in the low frequency range, which is particularly affected by the discarding of many data points. This shows distinct temporal correlations, and therefore no improvement can be achieved by averaging over time.

According to one variant, the section is arranged on the neuromodulation ASIC (application specific integrated circuit) with 32 bidirectional channels. Said neuromodulation ASIC is designed to be very flexible and can thus be used in many different application scenarios.

Furthermore, the detection section can be designed to perform signal filtering, in particular high-pass filtering, which is adjustable according to the predetermined scheme. The signal filtering is thus adaptable to the data rate of the data stream (data points per unit of time) after the reduction of the data rate by the data rate reduction section.

### BRIEF DESCRIPTION OF THE DRAWING

The invention and exemplary embodiments are described in more detail with reference to the drawing. In the drawings:

FIG. 1 shows an exemplary embodiment of the invention; and

FIG. 2 shows the arrangement and function of the data reduction section.

### DETAILED DESCRIPTION

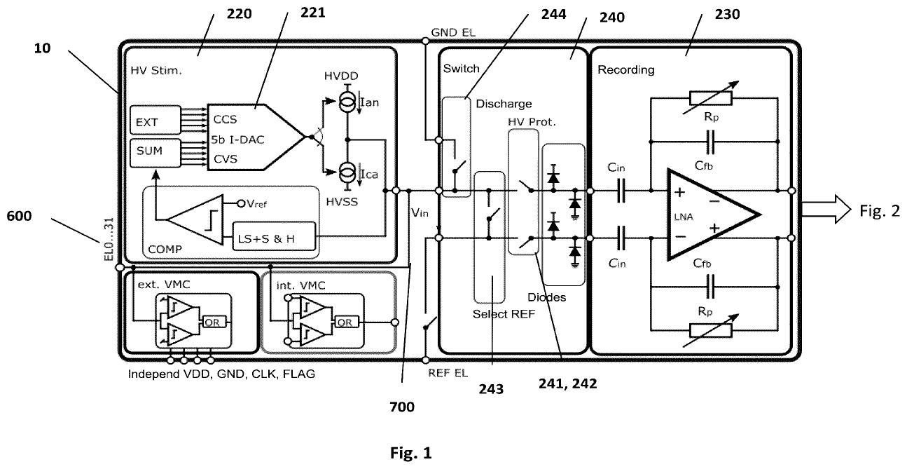

FIG. 1 illustrates a neuroimplant according to an exemplary embodiment of the invention, in which the data rate reduction section is realized.

The neuroimplant comprises at least one detection section 230 for detecting signals from the brain region and is divided into a plurality of similarly designed channels 10; a typical number is 32 channels. In FIG. 1, only one channel 10 is shown. This neuroimplant is therefore a neuromodulator or closed-loop neuroimplant.

The electrode section 600 comprises a number of electrodes and is connected to the at least one detection section 230. The number of electrodes is generally at least as large as the number of channels 10.

The neural implant according to one variant additionally has at least one stimulation section 220 for generating stimulation voltages for the brain region and an electrode section 600 connected to the stimulation section 220 for contacting the brain region. The electrode section 600 having at least one electrode is coupled to the stimulation section 220 via a connection 700 for transmitting the stimulation signals.

Each channel 10 is associated with or can be associated with exactly one electrode of the electrode section 600, a stimulation section 220 and a detection section 230.

Each channel 10 (according to the variant) is bi-directionally, i.e., can conduct electrical signals and other electrical variables in two directions. In the one direction, stimulation signals generated by the at least one stimulation section 220 are conducted to the respective electrode, in the opposite direction, neuronal signals from the brain region, which are detected via the electrode of the electrode section 600, are conducted to the at least one detection section 230.

The stimulation section 220 of each channel 10 comprises a digital-to-analog converter (DAC) 221 for generating analog stimulation signals from externally supplied digital control signals. The DAC 221 can be a 5-bit current DAC with a high-voltage push-pull output stage. The high voltage is, for example, 18 V.

The stimulation section 220 may be configured in various modes. In a first mode it generates current-controlled stimulation signals (CCS), in a second mode it generates voltage-controlled stimulation signals (CVS). In the second mode, the DAC 221 and the high-voltage output are embedded in a DSM-based feedback loop.

In the exemplary embodiment, the stimulation section 220 has an increased dynamic range by scaling the current mirror gain options. In the exemplary embodiment, the dynamic range is 66 dB, with stimulation currents of 5 μA to 10 mA.

The stimulation section 220 has flexible signal shape generation which is based on a state machine with sequential execution of stimulation commands. Said flexible signal shape generation is expanded by programmable loops within the command panel, enabling extended repeated implementations of waveforms and commands.

The entire neuroimplant can be controlled by a microcontroller unit (MCU, not illustrated).

The neuroimplant further comprises a switch matrix section 240 arranged between the electrode section 600 and the at least one detection section 230. The switch matrix section 240 comprises switching devices 241, 242 for each channel 10 for coupling the electrode of each channel 10 to the detection section 230 of the channel 10 or decoupling it therefrom. In addition, the switch matrix section 240 comprises switching devices 243 with which each electrode can be connected to ground GND. In this way, the relevant electrode can be discharged, for example.

Furthermore, the switch matrix section 240 comprises switching devices 244 with which an electrode (or multiple electrodes) can be connected as reference electrode REF. If the electrode is connected as reference electrode REF, it can be coupled to the reference input, for example the inverting input (−) of each detection section 230, while it can be decoupled from the measurement input, for example the non-inverting input (+) of the relevant detection section 230. In this way, the potential REF of the reference electrode can be applied to each detection section 230 as the common reference potential.

This feature is frequently requested in practical applications because the characteristic of implanted electrodes prior to implantation is unknown and, moreover, can vary over time, which makes it disadvantageous to define a specific electrode as reference electrode a priori.

Thus, the switch matrix section 240 is adaptable in such a way that the reference electrode REF is not simultaneously used as a charge sink for the passive discharge of electrodes after the stimulation, because this can lead to artefacts and charging of the reference electrode REF. Instead, an electrode different from the reference electrode is used as ground electrode GND for discharging. This also provides a better reference when detecting and recording the neuronal signals.

Each channel 10 is digitally controlled by a state machine which is programmable by means of SPI communication (SPI=Serial Peripheral Interface).

Furthermore, the at least one detection section 230 is programmable, whereby it is made possible to record either local field potential (LPFs), action potential (APs) or both bands with selectable optional amplification and bandwidth settings.

At least one digitization section 800, specifically an analog-to-digital converter (ADC, I-DSM 1, I-DSM 2), is provided for digitizing the detected analog neuronal signals and for generating at least one data stream (via SPI 850) therefrom. In an exemplary embodiment, two 16-bit ADCs are provided; each ADC can be coupled to 16 channels. Each ADC can be periodically connected to the individual channels in time-division multiplexing, which digitize the signals detected in this way.

The sampling frequency of the ADC is usually constant, e.g. 20 kHz. However, this exceeds the required data rate if only LFP signals are to be recorded, which leads to an unnecessarily high workload on the MCU which is to further process the data streams. This is because said MCU would then have to downsample the data streams in order to avoid excessive power consumption in the SPI Bluetooth connection outside the body.

Therefore, a section 900 for reducing the data rate of the digitized data streams is provided, which section is designed to adapt the digital data streams according to the bandwidth of the SPI interface. For this purpose, the data reduction section 900 can be programmed in such a way that it discards n data points from each sequence of m data points of the data stream of a channel and only allows the other data points to pass unchanged; in this case, m and n are natural numbers.

Additionally or alternatively, individual channels can be completely deactivated, i.e., the data points of the data stream in question can be completely discarded.

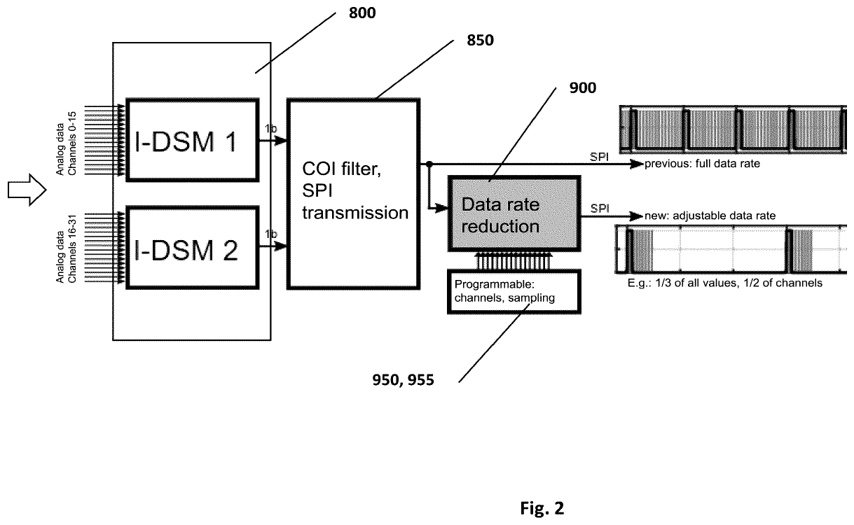

FIG. 2 illustrates the arrangement and function of the data reduction section.

A data stream with full data rate, i.e., without data reduction by discarding data points, is shown at the top right of the figure, below with reduction.

According to the invention, data points (or samples) may be suppressed according to the respectively valid reduction scheme, while the non-suppressed data points are forwarded unchanged. In the example, the data streams of half of the channels 10 are discarded, while only every third point of the remaining data streams is transmitted, i.e., in each case two of three data points are discarded (m=3, n=2).

A first register 950 can be provided for selecting the data stream or data streams (channel/channels 10) which are to be completely suppressed. By setting this, for example, 32-bit register, it is determined individually for each channel whether the detected data stream is output (“1”) or discarded (“0”).

In order to define whether individual data points of a data stream are to be discarded, a second register 955 can be provided. By setting the, for example, 5-bit register, a number is defined that determines how many available data points per channel are discarded between two data points that are actually sent. For example, a 1 (binary “00001”) means that one data point is discarded between two output data points—thus only every second data point leaves the chip, and the data rate is halved accordingly.

Transmitting only a part of the samples may seem like a primitive measure, because averaging over a plurality of samples could suppress noise by downsampling. However, this is only true for white noise, but not for correlated flicker noise, which is predominant in LPF signals. Because downsampling is not feasible in the AP range dominated by white noise due to bandwidth requirements, there is no significant benefit there either. Thus, downsampling can be completely omitted.

The reduction of the data rate is therefore achieved by a section 900 which is arranged on the ASIC together with the other sections of the neuroimplant. The data rate can therefore be reduced even before the data stream leaves the neuroimplant. The (external) MPU is therefore relieved of the object of protecting and the communication connection is spared in terms of the required bandwidth.

Furthermore, the detection section 230 may be designed to perform signal filtering, in particular high-pass filtering, which is adjustable according to the predetermined scheme. The signal filtering is thus adaptable to the data rate of the data stream (data points per unit of time) after the reduction of the data rate by the data rate reduction section.

What is claimed is:

1. A neuroimplant for the brain region, comprising:
at least one detection section for detecting signals from the brain region,
an electrode section having at least one electrode which can be coupled to the detection section and can be arranged in the brain region and is designed for electrically contacting the brain region,
at least one digitization section for generating at least one digital data stream from the signals detected by the at least one detection section,
wherein the at least one data stream in each case comprises a sequence of data points,
and a data reduction section designed to discard data points in the sequence of data points from the at least one data stream according to a predetermined scheme.

2. The neuroimplant according to claim 1, wherein the scheme comprises discarding all data points of a data stream.

3. The neuroimplant according to claim 1, wherein the scheme comprises regularly discarding a predetermined number of data points from the at least one data stream.

4. The neuroimplant according to claim 1, wherein the scheme comprises discarding n data points from each sequence of m data points of a data stream, where m and n are natural numbers.

5. The neuroimplant according to claim 1, wherein a first register is provided which contains, for each of the at least one data stream, a value representative of whether or not all data points of the respective data stream are discarded.

6. The neuroimplant according to claim 5, wherein a second register is provided which contains a value representative of discarding n data points from each sequence of m data points of at least one data stream.

7. The neuroimplant according to claim 6, wherein at least one of the first and second registers is programmable.

8. The neuroimplant according to claim 1, wherein the at least one detection section, the at least one digitization section and the data reduction section are arranged on a common semiconductor substrate.

9. The neuroimplant according to claim 1, wherein the at least one detection section, the at least one digitization section and the data reduction section are implemented as an ASIC chip.

10. The neuroimplant according to claim 1, further comprising:
at least one stimulation section for generating stimulation signals for the brain region.

11. The neuroimplant according to claim 1, wherein the neuroimplant is divided into a number of channels, each channel comprising a simulation section, a detection section and an electrode of the electrode section.

12. The neuroimplant according to claim 1, wherein the detection section is designed to perform signal high-pass filtering, which is adjustable according to the predetermined scheme.

13. The neuroimplant according to claim 1, further comprising a switch matrix section arranged between the electrode section and the at least one detection section, wherein the switch matrix section comprises switching devices for coupling the electrode of each channel to the detection section of the channel or for decoupling it therefrom.

14. The neuroimplant according to claim 12, wherein the switch matrix section comprises switching devices by means of which an electrode can be respectively connected to an earth point.

15. The neuroimplant according to claim 13, wherein the switch matrix section comprises switching devices by means of which a predetermined electrode can be connected as a reference electrode for the at least one detection section.
